(19) United States
(12) Patent Application Publication
(10) Pub. No.: US 2025/0380883 A1
(43) Pub. Date: Dec. 18, 2025

(54) METHOD FOR DETERMINING A MASKING INTENSITY IN A CONTRALATERAL EAR AND ASSOCIATED ELECTRONIC DEVICE

(71) Applicant: MY MEDICAL ASSISTANT, Reims (FR)

(72) Inventor: Nicolas WALLAERT, Reims (FR)

(73) Assignee: MY MEDICAL ASSISTANT, Reims (FR)

(21) Appl. No.: 18/835,874

(22) PCT Filed: Feb. 6, 2023
(86) PCT No.: PCT/EP2023/052761

§ 371 Date: Feb. 11, 2025

(30) Foreign Application Priority Data
Feb. 8, 2022 (FR) FR2201114

(51) Int. Cl. A61B 5/12; A61B 5/00

(52) U.S. Cl. CPC A61B 5/123; A61B 5/0051

(57) ABSTRACT

An audiometry test method comprising the following steps:
Simultaneous application of a test sound to an ear (210) with a test intensity, and of a masking sound, with a masking intensity, to a contralateral ear (220) to mask the test sound,
   the process being characterized in that it comprises the following steps:
Repeat the following steps:
   Determining a current Rinne,
   Determining that the current Rinne is compatible, if there is a current intensity, such that if the masking intensity is equal to the current intensity and the Rinne of the (210) or contralateral (220) ear is equal to the current Rinne, then the test sound is not perceived by the contralateral ear (220) and the masking sound does not prevent the perception of the test sound by the ear (210),
Determining masking intensity from maximum compatible current Rinne.

The invention concerns pure-tone audiometry.

Audiometry testing methods make it possible to measure discrete values of hearing thresholds, in air transmission mode and in bone transmission, to form an audiogram over a sound range extending, for example, from 125 to 8000 Hertz (as per standards, Hertz is referred to as Hz in the following) in air transmission mode and from 250 to 6000 Hz in bone transmission mode.

A series of test sounds is applied to the ear under test, for example via air-conduction headphones or a bone-conduction vibrator. The patient is asked to press an answer button as soon as he hears a test sound.

The audiometry test can be flawed if the test sound is perceived by the contralateral ear (that is, the ear not being tested). For this reason, the test sound must be masked by a masking sound applied to the contralateral ear in air-conduction, usually via headphones. However, the masking sound must not prevent the test sound from being perceived by the ear under test. Estimation of the masking sound meeting these constraints is based on the Air-Bone gap or Rinne (that is, the difference (in dB) between air-conduction and bone-conduction) and (properties of) transcranial transfer (transmission of the test sound to the contralateral ear). To estimate the Rinne, in the state of the art, we first perform an air-conduction audiogram and an unmasked bone-conduction audiogram, before performing the same tests while masking the contralateral ear with the Rinne thus obtained. Performing the first two audiograms lengthens the audiometry test time, while providing an imprecise estimate of the Rinne, since no masking is applied when performing these audiometry tests.

To remedy these drawbacks, the invention relates to an audiometry test method comprising the following steps:
Simultaneous application (S50) of a test sound to a patient's test ear (210) having a test intensity, and of a masking sound, having a masking intensity, to a contralateral ear (220) to mask the test sound,
the process being characterized in that it comprises at least one of the following two pairs of steps:
The first pair of steps:
   Determination of a theoretical Rinne from a theoretical transcranial transfer,
   Determination of masking intensity from the theoretical Rinne.
The second pair of steps:
   Determination of a theoretical transcranial transfer from a theoretical Rinne,
      Determination of masking intensity from the theoretical transcranial transfer.

For example, in air-conduction, the theoretical transcranial transfer is between 40 dB and 80 dB, for example, equal to 50 dB (as per standards, decibels are usually referred to as dB in the following). In bone-conduction, the theoretical transcranial transfer is comprised between −10 dB and 20 dB. The origin of these values is given in Appendix 1 and 2.

For example, the theoretical Rinne is between 0 and 70 dB.

In this way, masking is determined without the need for prior bone-conduction and air-conduction audiometry. The invention thus makes it possible to speed up audiometric testing of a patient. The masking intensity is determined from a theoretical Rinne obtained from a theoretical transcranial transfer (i.e., a hypothetical value of the transcranial transfer) or is determined from a theoretical transcranial transfer obtained from a theoretical Rinne (i.e., a hypothetical value of the Rinne). Alternatively, the masking intensity is determined by testing several Rinne or transcranial transfer values. The invention thus also relates (independently of the above invention) to an audiometry testing method comprising the following steps, or according to one embodiment (of the above invention), the method comprises the following steps:
Simultaneous application (S50) of a test sound to a test ear (210) of a patient (200) comprising a test intensity, and of a masking sound, comprising a masking intensity, to a contralateral ear (220) to mask the test sound,
the method being characterized in that it comprises at least one of the following two double steps (hence, potentially both double steps at the same time), the patient (200) comprising a transcranial transfer:
First double steps:
   Repeat the following (two) steps:
      Determining a current Rinne,
      Determining (S30) that the current Rinne is compatible, if there is a current intensity, such that if the masking intensity is equal to the current intensity and the Rinne of the test ear (210) or contralateral ear (220) is equal to the current Rinne, then the test sound is not perceived by the contralateral ear (220) and the masking sound does not prevent perception of the test sound by the test ear (210),
   Determination of a masking intensity from a maximum compatible current Rinne determined during the repetition step,
Second double steps:
   Repeat the following (two) steps:
      Determination of a current transcranial transfer,
      Determining (S30) that the current transcranial transfer is compatible, if there is a current intensity, such that if the masking intensity is equal to the current intensity and the patient's transcranial transfer (200) is equal to the current transcranial transfer, then the test sound is not perceived by the contralateral ear (220) and the masking sound does not prevent perception of the test sound by the tested ear (210),
   Determination of a masking intensity from a maximum compatible current transcranial transfer determined during the repetition step.

Repetition means that the above (two) steps can be performed two or more times (for the same test sound).

In one embodiment, the (two) steps are repeated, for the first double steps, until the current Rinne is compatible, or for the second double steps, until the transcranial transfer is compatible.

According to one embodiment, the step of determining a current Rinne comprises a step of decrementing the current Rinne by one step, the method further comprising a step of initializing the current Rinne to an initialization Rinne.

Alternatively, values of the current Rinne can be scanned in different ways (according to an increasing current Rinne, for example).

In one embodiment, the initialization Rinne is between 55 dB and 65 dB, preferably 60 dB. This initialization Rinne is higher than the actual Rinne of most patients.

In one embodiment, the step size is between 1 dB and 20 dB, preferably 5 dB.

In one example, if the maximum compatible current Rinne is below a minimum threshold, an alarm message is sent (and displayed).

The minimum threshold is, for example, between 0 dB and 45 dB, for example, equal to 40 dB. This minimum threshold value is determined from the results of a previously obtained Lateralization or Weber test. For example, the test process includes a Weber test, and if the Weber test lateralizes to the side of the better ear, we are dealing with a probable sensorineural hearing loss (i.e., originating from the inner ear). The minimum threshold can, for example, be between 0 and 30 dB, and for example, be equal to 20 dB, as the Rinne is not likely to be significant. On the other hand, if the Weber test is lateralized to the side of the bad ear, we are dealing with a probable conductive or mixed hearing loss (i.e., originating from a condition of the outer or middle ear). In this case, for example, the minimum threshold could be between 25 and 65 dB, and set at 40 dB, as it is unlikely to have a low enough Rinne.

Thus, the audiometry test according to the invention may comprise an interrogation of the patient to find out his better ear and a Weber test, and in which the minimum threshold is between 0 and 40 dB if the Weber test is lateralized on the side of the better ear and between 25 and 65 if the Weber test is lateralized on the opposite side of the better ear. For some patients, the actual Rinne may in fact be lower than the minimum threshold, and may even be zero (i.e., equal to 0 dB).

For example, the step of determining that the current Rinne (or theoretical Rinne) is compatible comprises the following steps:
Calculation from test intensity (and current Rinne) of an efficacy threshold for masking intensity, above which the test sound cannot be perceived by the contralateral ear,
Calculation from the test intensity (and current Rinne) of a no-overmasking threshold for masking intensity below which the masking sound does not prevent perception of the test sound (i.e., below which the test sound is perceived in the presence of the masking sound) by the ear under test,
A step to compare the efficacy criterion and the no-overmasking thresholds,
the current Rinne (or theoretical Rinne) being compatible if the efficacy threshold is less than or equal to the no-overmasking threshold.

For example, as the audiometry test is performed in air-conduction (i.e., the test sound is applied to the air-conducting ear under test, for example, using air-conduction headphones, and the masking sound is applied to the contralateral air-conducting ear, for example, using air-conduction headphones), the calculation of the no-overmasking threshold is based on the current Rinne.

For example, in air-conduction, the no-overmasking threshold (SR) is equal to:

SR=IT−current Rinne_{tested ear}+TTC−RSBR, where
SR is the no-overmasking threshold (in dB),
IT is the test intensity (in dB),
TTC is the transcranial transfer (in dB), (in other words, an assumption about the transcranial transfer, as this cannot be measured on the patient).
current Rinne_{tested ear} is the current Rinne of the tested ear (210).
RSBR is the signal-to-noise ratio (so that the masking sound applied to the contralateral ear does not prevent the test sound from being heard in the test ear).

For example, in bone-conduction (i.e., the test sound is applied via bone-conduction to the test ear, for example, using a vibrator, and the masking sound is applied via air-conduction to the contralateral ear, for example, using air-conduction headphones), the no-overmasking threshold (SR) is equal to:

SR=IT+TTC−RSBR, with the same notations as above.

For example, the signal-to-noise ratio RSBR is less than 10 dB, for example, equal to 0 dB.

In one embodiment, the efficacy threshold is calculated based on the current Rinne.

For example, in air-conduction, the efficacy threshold (SE) is equal to:

SE=IT−TTC+RSBE+current Rinne_{contro}, with the same notations as above, and where
SE is the efficacy threshold (in dB),
current Rinne_{contro} is the current Rinne of the contralateral ear,
RSBE is the signal-to-noise ratio (so that the masking sound covers the test sound in the contralateral ear).

For example, in bone-conduction, the efficacy threshold (SE) is equal to:

SE=IT+RSBE+current Rinne_{contro}, with the same notations as above.

For example, the signal-to-noise ratio RSBE is greater than 15 dB, for example equal to (+)20 dB.

In air-conduction, the transcranial transfer is between 40 dB and 80 dB, for example, 50 dB. In bone-conduction, the transcranial transfer is between −10 dB and 20 dB. The value of the transcranial transfer can be adjusted as a function of frequency, for both air-conduction and bone-conduction.

According to an embodiment, the Rinne of the contralateral ear can take on (in other words, be replaced by) a fixed value equal to:
A value depending on the threshold of an air-conduction audiogram of the contralateral ear, if this is (previously) known (for the frequency of the test sound, and this previous audiometry test can form part of the process according to the invention), for example, the threshold of the audiogram divided by a value greater than 2, for example, by 3 if the Weber test is lateralized to the side of the better ear, or alternatively by a value between 1 and 4, for example, equal to 1.5 if the Weber test is lateralized to the side of the worse ear, or
A Rinne value for the contralateral ear estimated from a previous audiometry test (and this previous audiometry test may be part of the process according to the invention) (for example, if the choice of test sounds is made by a Gaussian process, the Rinne value of the contralateral ear can be obtained from the initialization of this Gaussian process, which can be carried out for both ears in bone- and air-conduction, before starting the selection of test sounds by the Gaussian process for both ears in bone- and air-conduction, or from the current Rinne estimate when the Gaussian process is engaged).

The repeat step is not performed on the Rinne of the contralateral ear in this case (but on the Rinne of the ear under test).

In one embodiment, the Rinne (for example, the theoretical Rinne) cannot exceed a maximum threshold, for example, 60 dB. For example, if the air-conduction audiogram function value is above the maximum threshold, the (theoretical) Rinne is reduced to the high threshold.

According to an embodiment, during the masking intensity determination step, the Rinne current takes on the value of the maximum compatible current Rinne:
If the efficacy threshold and the no-overmasking threshold are equal, then the masking intensity takes the efficacy threshold as its value,
If the efficacy threshold is lower than the no-overmasking threshold, then the masking intensity takes on a value intermediate between the efficacy and no-overmasking thresholds (included), for example, the arithmetic mean of the no-overmasking threshold and the efficacy threshold.

According to a variant, the theoretical Rinne can be determined in bone-conduction, by solving the equation:

IT+RSBE+Theoretical Rinne=IT+TTC−RSBR. (i.e., Theoretical Rinne=TTC−RSBE−RSBR).

By construction, this theoretical Rinne is compatible, of course.

Alternatively, in the same way, a theoretical transcranial transfer can be determined in bone-conduction, by solving the same equation, based on a theoretical Rinne (in other words, i.e., a hypothetical value of the Rinne).

The masking intensity is then set, for example, to the efficacy threshold (SE), as defined above.

According to a variant, the step of determining that the current Rinne is compatible comprises the following steps:
Calculate an intensity sufficient to mask the test sound in the contralateral ear from the test intensity (and possibly the current Rinne),
Calculation of the masking sound's impact on the ear to be tested, based on sufficient intensity (and possibly current Rinne), the current Rinne being compatible if the masking sound is less than the test intensity plus a signal-to-noise ratio, for example, equal to 0 dB.

For example, the masking intensity is capped at a high threshold, for example, equal to 85 dB. For example, if the air-conduction audiogram function value is above threshold, the Rinne is brought back to the high threshold.

Instead of determining a theoretical Rinne from a transcranial transfer assumption, a theoretical transcranial transfer can be determined from a Rinne assumption without departing from the scope of the invention, in a manner analogous to that described in this application for determining the theoretical Rinne. The features according to the invention for the case where a theoretical transcranial transfer is determined from an assumption on the Rinne are analogous to the case where a theoretical Rinne is determined from an assumption on the transcranial transfer, which is why these features are not detailed here.

According to one embodiment:
The audiometry test is performed in air-conduction, and the ear tested (first) is the better ear (comprising an opposite ear), the test method then comprising an audiometry test of the opposite ear of the ear tested in air-conduction (according to the invention), wherein
The audiometry test is carried out in bone-conduction, and the ear tested is the ear in which a Weber test has been lateralized (the method according to the invention may comprise, for example, the Weber test), the test method then comprising an audiometry test of the ear opposite to the ear tested in bone-conduction (according to the invention).

So, we start with the better ear in air-conduction, and with the ear that probably has the greatest Rinne in bone-conduction (the ear towards which the Weber test was lateralized to).

The process according to the invention can be carried out (in other words, implemented) by an electronic audiometry testing device. The electronic device may comprise a central electronic unit (for example, included in a cell phone or touch-screen tablet) and headphones or inserts, or loudspeakers for applying sounds to the ear and masking the contralateral ear in air transmission. For bone transmission, one or more vibrators are used. Information on whether a sound is heard or not can be acquired by pressing a button when a sound is heard, or by voice command or image detection.

The invention therefore also relates to an electronic audiometry testing device configured to implement the steps of the process according to the invention.

The invention also relates to a computer program comprising instructions, executable by a microprocessor or microcontroller, for implementing the method according to the invention.

The features and benefits of the electronic device and the computer program are identical to those of the process, so they are not repeated here.

An element such as an electronic audiometry test device, central processing unit or other element is “configured to” perform a step or operation, by virtue of the fact that the element comprises means for (in other words, “is configured to” or “is adapted to”) performing the step or operation. These are preferably electronic means, such as a computer program, stored data and/or specialized electronic circuits.

When a step or operation is carried out by such an element, this generally implies that the element has means for (in other words, “is shaped to” or “is adapted to”) carrying out the step or operation. These may include electronic means, such as a computer program, stored data and/or specialized electronic circuits.

Other features and advantages of the present invention will become clearer on reading the following detailed description including modes of realization of the invention given by way of non-exhaustive examples and illustrated by figures in the Appendix:

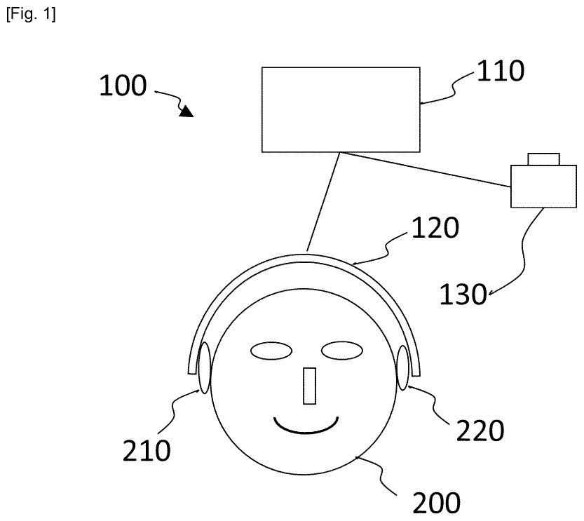

FIG. 1 shows an electronic device according to one embodiment of the invention.

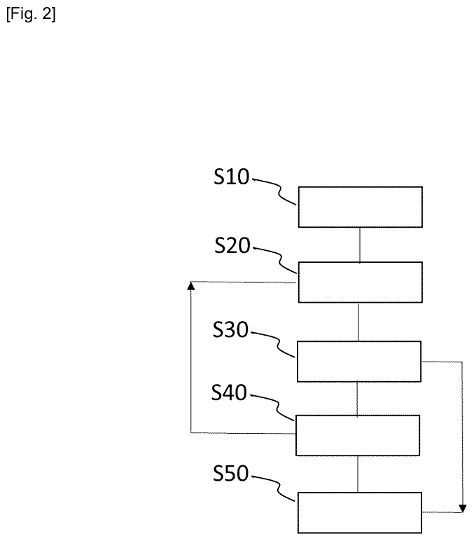

FIG. 2 shows the process according to the invention, in one example embodiment, implemented by the electronic device shown in FIG. 1.

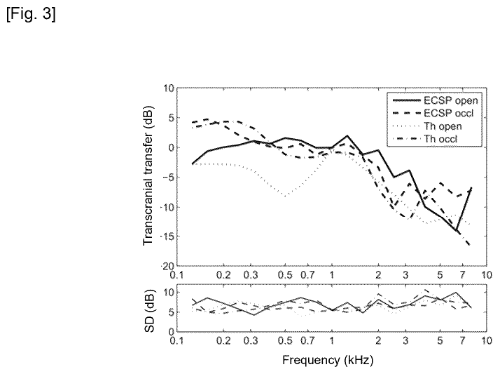

FIG. 3 Mean estimates of transcranial transfer obtained by ECSP measures for open (solid line) and occluded (dotted line) ear canals, and obtained by BC (bone-conduction) auditory thresholds for open, named Th open (dotted line) and occluded, named Th occluded (dashed line) ear canals. Standard deviation results, called SD, use the same symbols for the legend.

### DETAILED DESCRIPTION OF AN EMBODIMENT OF THE INVENTION

Referring to FIGS. 1 and 2, for a bone-conduction audiometry test, in order to determine the masking intensity for a test intensity, in step S10, the central unit 110 initializes in memory a current Rinne_{contro} at 60 dB, and in step S20, the central unit 110 calculates SE and SR such that:

SR=IT+50, and

SE=IT+20+current Rinne_{contro}, where
SE is the efficacy threshold (in dB), SR is the no-overmasking threshold (in dB),
IT is the test intensity (in dB),
current Rinne_{contro} is the current Rinne of the contralateral ear.

We therefore assume a theoretical transcranial transfer of 50 dB.

In step S30, SE and SR are compared by the central unit 110, if SR=SE then the current Rinne_{contro} is the maximum compatible current Rinne and the masking intensity is equal to SR (and SE). If SR=SE or SE<SR, then the current Rinne_{contro} is the maximum compatible current Rinne, and the masking intensity is equal to the arithmetic mean of the no-overmasking threshold and the efficacy threshold. However, if the masking intensity thus determined is greater than 85 dB, it is reduced to 85 dB. If SR=SE or SE<SR, then the process continues to step S50. In step S40, if SE>SR, then the current Rinne_{contro} is decremented by 5 dB, and the process continues to step S20.

In step S50, the central unit controls the emission of a test sound in the test ear 210, the sound having a test intensity, and of the masking sound in the contralateral ear 220 via the headset 120. If the current Rinne_{contro} (that is, maximum compatible Rinne) is less than 40 dB, then an alert message is transmitted to a display (not shown for display). The alert message may, for example, read: “Audiometry uncertain—Rainville ipsilateral masking audiometry recommended to confirm”.

In air-conduction, steps S10 to S50 can be carried out, but with:

SR=IT−current Rinne_{test ear}+50, and

SE=IT−50+20+current Rinne_{contro}, where
SE is the efficacy threshold (in dB), SR is the no-overmasking threshold (in dB),
IT is the test intensity (in dB),
current Rinne_{contro} is the Rinne current of the contralateral ear,
current Rinne_{test ear} is the current Rinne of the tested ear.

The current Rinne_{test ear} can be initialized in step S10 and decremented in step S30, simultaneously with the current Rinne_{contro}, in a loop nested with the one where the current Rinne_{contro} is decremented or in a different loop.

The current Rinne_{contro} can be replaced by one of the following fixed values:
A value based on the threshold of an air-conduction audiogram of the contralateral ear if this is (previously) known (for the frequency of the test sound, and this previous audiometry test can be part of the process according to the invention), for example, the threshold of the audiogram divided by 3 (for example, if the Weber test lateralizes to the side of the better ear) or 1.5 (if the Weber lateralizes to the side of the worse ear), or
A Rinne value for the contralateral ear estimated from a previous audiometry test (and this previous audiometry test can be part of the process according to the invention) (for example, if the choice of test sounds is made by a Gaussian process, the value of the Rinne of the contralateral ear can be obtained from the initialization of this Gaussian process, which can be performed for both ears in bone- and air-conduction, before starting the selection of test sounds by the Gaussian process for both ears in bone- and air-conduction, or from the Rinne resulting from the threshold estimation of the Gaussian process).

The Rinne initialization and decrement steps for the contralateral ear are not performed in this case (but the Rinne steps for the test ear are).

Alternatively, the central unit can determine a masking intensity, for example from a 10 dB bone-conduction test tone as follows:

The test sound arrives in the contralateral ear at 10 dB. The central unit determines that 90 dB is sufficient to mask (in air transmission) in the contralateral ear, with a contralateral Rinne assumption of 60 dB (the signal-to-noise ratio for the masking sound to mask the test sound being set at 20 dB). With a transcranial transfer of 50 dB, the test ear receives 40 dB of the masking sound, preventing the test sound from being heard in the test ear (the signal-to-noise ratio is equal to −30 dB).

With a contralateral Rinne assumption of 40 dB, 70 dB is sufficient to mask the test sound in the contralateral ear. With a transcranial transfer of 50 dB, the test ear receives 20 dB of masking sound, preventing the test sound from being heard in the test ear (the signal-to-noise ratio is equal to −10 dB).

With a contralateral Rinne assumption of 30 dB, 60 dB is sufficient to mask the test sound in the contralateral ear. With a transcranial transfer of 50 dB, the test ear receives 10 dB of masking sound, which does not prevent the test sound from being heard in the test ear (the signal-to-noise ratio is equal to 0 dB). The masking intensity is therefore 60 dB.

If the Weber was lateralized to the better ear, the current Rinne_{contro} (i.e., the maximum compatible current Rinne) is greater than the Rinne threshold value (e.g., 20 dB), and no alert message is displayed. If, however, the Weber was lateralized to the deafer ear, the current Rinne_{contro} (i.e., maximum compatible current Rinne) is less than 40 dB, so an alert message is transmitted to a display (not shown for display). The alert message may, for example, read: “Audiometry uncertain—Rainville ipsilateral masking audiometry recommended to confirm”.

According to an embodiment, the audiometry test for a patient is performed in the following chronological order:
Interrogating the patient to find out about his better ear
Weber test: Binaural or frontal bone-conduction stimulation
Note to which ear the sound is lateralized to,
Air-conduction test of the better ear,
Air-conduction test of the other ear,
Bone-conduction test of the ear to which the sound is lateralized,
Bone-conduction test of the other ear.

Questioning the patient helps determine what is a priori his better ear.

For example, during the testing process according to the invention, a Gaussian process can be implemented by the central unit 110 to determine the sounds to be tested, for example as described in the article by Schlittenlacher J, Turner R E, Moore B C J. “Audiogram estimation using Bayesian active learning” (J Acoust Soc Am. 2018; 144(1):421).

The process according to the invention can be carried out (in other words, implemented) by an electronic device 100 of audiometry testing. The electronic device 100 may comprise a central unit 110 and air-conduction headphones 120 for applying sounds to the ear 210 and masking the contralateral ear 220 in air transmission of a patient 200. In bone transmission, one or more vibrators (not shown) are used for the tested ear 210 in combination with air-conduction headphones for masking the contralateral ear 220 in air transmission. Information as to whether a sound is heard or not can be acquired by means of a response button 130, which the patient presses when a sound is heard.

### APPENDIX 1: ORIGIN OF TRANSCRANIAL TRANSFER VALUES IN AIR-CONDUCTION (AC)

The following tables show the minimum interaural attenuations (minimum observable transcranial transfers) for THD 39 headphones (airborne) and audiometric inserts (airborne).
Katz, J., Lezynski, J. (2002). Clinical Masking. In J. Katz, ed, Handbook of Clinical Audiology, Williams and Wilkins, Baltimore.
Munro, K. J., Agnew, N. A comparison of inter-aural attenuation with the Etymotic ER-3A insert earphone and the Telephonics TDH-39 supra-aural earphone. Br J Audiol 1999; 33:259-262.
Hall, J W., Mueller, H G. (1997). The audiologists' desk reference, Volume I, Singular Publishing Group, San Diego

Min IA (over-the-ear headphones: TDH-39),
specific for each audiometric frequency

| Hz | dB | Bibliographical reference |
|---|---|---|
| 125 | 35 | Katz & Lezynski, (2002) |
| 250 | 48 | Munro & Agnew, BJA (1999) |
| 500 | 44 | Munro & Agnew, BJA (1999) |
| 750 | 40 | N/A - fulfill traditional approach |
| 1000 | 48 | Munro & Agnew, BJA (1999) |
| 1500 | 40 | N/A - fulfill traditional approach |
| 2000 | 44 | Munro & Agnew, BJA (1999) |
| 3000 | 56 | Hall J. W. III & Mueller G. H. III/Munro & |
| | | Agnew, BJA (1999) |
| 4000 | 50 | Katz J/Munro & Agnew, BJA (1999) |
| 6000 | 44 | Hall J. W. III & Mueller G. H. III/Munro & |
| | | Agnew, BJA (1999) |
| 8000 | 42 | Katz J/Munro & Agnew, BJA (1999) |

Min IA (audiometric inserts), specific
for each audiometric frequency

| Hz | dB | Bibliographical reference |
|---|---|---|
| 125 | 60 | N/A - traditional value |
| 250 | 72 | Munro & Agnew, BJA (1999) |
| 500 | 64 | Munro & Agnew, BJA (1999) |
| 750 | 60 | N/A - traditional value |
| 1000 | 58 | Munro & Agnew, BJA (1999) |
| 1500 | 60 | N/A - traditional value |
| 2000 | 56 | Munro & Agnew, BJA (1999) |
| 3000 | 58 | Munro & Agnew, BJA (1999) |
| 4000 | 72 | Munro & Agnew, BJA (1999) |
| 6000 | 54 | Munro & Agnew, BJA (1999) |
| 8000 | 62 | Munro & Agnew, BJA (1999) |

### APPENDIX 2: ORIGIN OF TRANSCRANIAL TRANSFER VALUES IN BONE-CONDUCTION (BC)

1. An audiometry test method comprising the following steps:
Simultaneous application (S50) of a test sound to a test ear (210) of a patient (200) comprising a test intensity, and of a masking sound, comprising a masking intensity, to a contralateral ear (220) to mask the test sound,
the process being characterized in that it comprises at least one of the following two pairs of steps:
The first pair of steps:
Determination of a theoretical Rinne from a theoretical transcranial transfer,
Determination of masking intensity from the theoretical Rinne
The second pair of steps:
Determination of a theoretical transcranial transfer from a theoretical Rinne,
Determination of masking intensity from the theoretical transcranial transfer.

2. An audiometry test method according to the preceding claim further comprising at least one of the following two double steps, the patient (200) comprising a transcranial transfer:
First double steps:
Repeat the following steps:
Determining a current Rinne,
Determining (S30) that the current Rinne is compatible, if there is a current intensity, such that if the masking intensity is equal to the current intensity and the Rinne of the test ear (210) or contralateral ear (220) is equal to the current Rinne, then the test sound is not perceived by the contralateral ear (220) and the masking sound does not prevent perception of the test sound by the test ear (210),
Determination of a masking intensity from a maximum compatible current Rinne determined during the repetition step,
Second double steps:
Repeat the following steps:
Determination of a current transcranial transfer,
Determining (S30) that the current transcranial transfer is compatible, if there is a current intensity, such that if the masking intensity is equal to the current intensity and the patient's transcranial transfer (200) is equal to the current transcranial transfer, then the test sound is not perceived by the contralateral ear (220) and the masking sound does not prevent perception of the test sound by the tested ear (210),
Determination of a masking intensity from a maximum compatible current transcranial transfer determined during the repetition step.

3. An audiometry test method according to the preceding claim, wherein the audiometry test method comprises the first double steps, and wherein the step of determining a current Rinne comprises a step of decrementing (S40) the current Rinne by one step, the method further comprising a step of initializing (S10) the current Rinne to an initialization Rinne.

4. An audiometry test method according to any one of claim 2 or 3 wherein the audiometry test method comprises the first double steps, and wherein if the maximum compatible current Rinne is below a minimum threshold, an alarm message is sent.

5. An audiometry test method according to any one of claims 2 to 4 wherein the audiometry test method comprises the first double steps, and wherein the step of determining that the current Rinne is compatible comprises the following steps:
Calculation of an efficacy threshold for the masking intensity above which the test sound cannot be perceived by the contralateral ear (220), based on the test intensity,
Calculation of a no-overmasking threshold for the masking intensity below which the masking sound does not prevent perception of the test sound by the ear under test (210), based on the test intensity,
A step to compare the efficacy threshold and the no-overmasking threshold,
the current Rinne being compatible if the efficacy threshold is less than or equal to the no-overmasking threshold.

6. An audiometry test method according to claims 2 to 5, the audiometry test being performed in air-conduction, wherein the audiometry test method comprises the first double steps, and wherein the calculation of the efficacy threshold is performed from the current Rinne.

7. An audiometry test method according to claim 2 or 6, wherein the audiometry test method comprises the first double steps, and wherein the threshold calculation is performed on the basis of the current Rinne.

8. An audiometry test method according to any one of claims 5 to 7 wherein the audiometry test method comprises the first double steps, and wherein, during the step of determining the masking intensity, the current Rinne taking as its value the maximum compatible current Rinne:
If the efficacy threshold and the no-overmasking threshold are equal, then the masking intensity takes the efficacy threshold as its value, or
If the efficacy threshold is lower than the no-overmasking threshold, then the masking intensity takes on an intermediate value between the efficacy threshold and the no-overmasking threshold.

9. An audiometry test method according to any one of claims 2 to 4 wherein the audiometry test method comprises the second double steps, and wherein the step of determining that the current Rinne is compatible comprises the following steps:
Calculate an intensity sufficient to mask the test sound in the contralateral ear (220) from the test intensity,
Calculation of the impact of the masking sound in the test ear (210) based on the intensity,
the current Rinne being compatible if the masking sound is less than the test intensity plus a signal-to-noise ratio.

10. An audiometry test method according to any one of claims 1 to 9 in which:
The audiometry test is performed in air-conduction, and the tested ear (210) is the better ear, the test method then comprising an audiometry test of the opposite ear (220) to the tested ear (210) in air-conduction, where
The audiometry test is carried out in bone-conduction, and the tested ear (210) is the ear towards which a Weber test was lateralized to, the test method then comprising an audiometry test of the opposite ear (220) to the tested ear (210) in bone-conduction.

11. Electronic audiometry testing device (100) configured to implement the steps of the test method according to any one of claims 1 to 10.

12. Computer program comprising instructions, executable by a microprocessor or microcontroller, for implementing the test method according to any one of claims 1 to 10.
